(19) United States
(12) Patent Application Publication
(10) Pub. No.: US 2004/0116506 A1
(43) Pub. Date: Jun. 17, 2004

(54) Use of levetiracetam for treating or preventing acute headaches

(76) Inventor: John Claude Krusz, Dallas, TX (US)

Correspondence Address: Patrick D. Kelly, #403, 11939 Manchester, St. Louis, MO, 63131, US

(21) Appl. No.: 10/600,818

(22) Filed: Jun. 20, 2003

Related U.S. Application Data
(60) Provisional application No. 60/390,317, filed on Jun. 20, 2002.

(51) Int. Cl. A61K 31/4015

(52) U.S. Cl. 514/424

(57) ABSTRACT

Levetiracetam, an N-type calcium channel antagonist drug, has been discovered to be highly effective in treating migraine headaches. When administered orally on a daily basis, it reduces the adverse traits (including frequency, severity, and duration) of migraines among chronic sufferers. When injected intravenously, it was highly effective in entirely curing or greatly reducing full-blown acute migraine headaches, even among patients who could not be treated adequately by triptans or any other known drugs.

### RELATED APPLICATION

This application claims the benefit, under 35 USC 119(e), of provisional patent application No. 60/390,317, filed on Jun. 20, 2002.

### BACKGROUND OF THE INVENTION

This invention is in the field of pharmacology and headache treatments, and more particularly relates to the use of an N-type calcium channel blocker drug, called levetiracetam, for treating patients who suffer from migraine headaches.

Levetiracetam is the S-enantiomer of a drug called piracetam, known since the 1960's and classified as a “nootropic” drug; this term refers to drugs that assertedly can enhance and improve cognition, memory, or other mental functioning.

Levetiracetam was developed more recently, and was approved in the U.S. in late 1999, for treating patients who suffer from epilepsy. When taken chronically (i.e., such as by taking one pill each day), it has been shown to reduce the frequency of epileptic seizures, in patients who suffer from frequent seizures, by roughly 15 to 30%. Since it is used for this purpose, levetiracetam is generally classified as an anti-convulsant drug. It is known to suppress activity, to some degree, at so-called “N-type” calcium channels (also called neuronal or high-voltage calcium channels). N-type calcium channels are present on the surfaces of neurons in the central and peripheral nervous systems, and they are heavily involved in the depolarization (also called “firing”) events that are involved in the transmission of signals, (impulses) from one neuron to another.

Levetiracetam is available in the U.S. (by prescription only), in orally ingested tablet form, under the trademark KEPPRA™, sold by UCB Pharma, Inc. (Smyrna, Ga.); it is also available in various other countries, including Great Britain. It appears to be a relatively benign drug with a very low side-effect profile, and is well tolerated over a wide dose range with no apparent toxicity.

The Applicant herein is a physician who specializes in the treatment of pain and acute headaches, and he treats numerous patients who suffer from migraine and/or cluster headaches. To the best of his knowledge and belief, levetiracetam was not previously tested or reported as being efficacious for treating migraines, by anyone, prior to his discovery of that apparent efficacy and utility.

However, as a physician who treats numerous patients who suffer from recurrent migraine and cluster headaches, he is aware that the “armamentarium” of known and safe drugs that are truly effective in treating or preventing acute migraines and cluster headaches is seriously limited, and does not offer adequate relief to all patients who need relief from such headaches. Instead, even the best currently-known drugs that are widely used to treat acute migraine or cluster headaches (such as various triptan drugs) are not effective in all patients, and many patients do not receive adequate relief from them. In addition, triptans and other drugs that are currently used for treating acute migraine or cluster headaches cannot be used safely in many patients who suffer from other medical problems or conditions in addition to their recurrent migraines. Therefore, improved drugs and additional options, for treating acute migraines and cluster headaches, are still badly needed, among major segments of the general public who suffer from acute recurrent primary headaches.

In the mid-1980's and early 1990's, there was substantial interest in various calcium channel blockers as potential agents for preventing or reducing migraine headaches, and quite a few candidate drugs that suppress activity at calcium channels (including verapamil, diltiazem, nifedipine, flunarizine, various drugs that block N-type calcium channels) were tested against migraine headaches, as described in articles such as Greenberg 1986, Andersson et al 1990, Kim 1991, and Montastruc et al 1992. However, the calcium channel blockers that showed moderate levels of efficacy (such as flunarizine) were plagued by unwanted side effects, and the ones that were not plagued by serious side effects generally were no better at reducing migraines than other known classes of drugs, such as beta-blockers, which are not highly effective in most patients. Therefore, in the absence of any better results, interest in the possible use of calcium channel blockers for migraine sufferers dropped off substantially, in the early 1990's, especially after the introduction of triptan drugs. There are few known reports of any additional testing of calcium channel blocker drugs, against migraines, after the early 1990's.

Nevertheless, acting on a hunch, the Applicant decided to test a few patients who suffer from recurrent migraine headaches, by placing them (with their informed consent) on daily oral administration of levetiracetam. In this small-scale open-label trial, his patients reported a significant reduction in the frequency and/or severity (intensity, duration, etc.) of their migraine headaches (as used herein, traits such as frequency, intensity, and duration of acute headaches are referred to as “adverse traits” of such headaches).

Accordingly, one object of this invention is to disclose that orally-administered levetiracetam is clinically effective in substantial numbers of patients (including substantial numbers of patients who cannot be treated adequately by triptans or any other known drugs), as a prophylactic agent that can reduce the frequency, intensity, duration, or other adverse traits of recurrent migraine headaches, and possibly in reducing other types of acute headaches as well, including cluster headaches, and recurrent acute tension-type headaches.

Another object of this invention is to disclose that levetiracetam, when injected intravenously, is also highly clinically effective in substantial numbers of patients (including substantial numbers of patients who cannot be treated adequately by triptans or any other known drugs), as an agent for treating acute migraine headaches (and possibly cluster, tension-type, or other acute headaches) that have fully or partially emerged.

A third object of this invention is to disclose an injectable form of levetiracetam, in an aqueous carrier liquid, as a safe and effective agent with an important medical use.

These and other objects of the invention will become more apparent through the following summary, description, and examples.

### SUMMARY OF THE INVENTION

Levetiracetam, an N-type calcium channel antagonist drug which is the S-enantiomer of a drug called piracetam, has been discovered to be highly effective in treating migraine headaches. When administered orally on a chronic daily basis, it reduced the adverse traits (including frequency, severity, and duration) of migraines among chronic sufferers. When injected intravenously in an aqueous carrier liquid, it was highly effective in entirely curing or greatly reducing full-blown acute migraine headaches, even among substantial numbers of patients who could not be treated adequately by triptans or any other known drugs. Injectable aqueous formulations, which previously have not been developed or made available, are also disclosed herein.

### DETAILED DESCRIPTION

As described above in the Background section, there has been essentially no active interest in using or testing calcium channel blockers as agents to treat or prevent migraine headaches, since the early 1990's. However, the Applicant herein became aware of a drug called levetiracetam, which is generally classified as an anti-convulsant drug. When taken chronically (i.e., usually on a once-a-day basis), it is effective in reducing the number and/or frequency of seizures, among people who suffer from epilepsy. It is available in the U.S., by prescription only, in orally ingested tablet form, under the trademark KEPPRA™, sold by UCB Pharma, Inc. (Smyrna, Ga.).

Since levetiracetam reportedly is a relatively benign drug with a low side-effect profile that is well-tolerated over a wide dosage range, with no apparent toxicity, the Applicant herein (a physician who specializes in treating chronic pain and recurrent headaches) decided to test a few patients who suffer from recurrent migraine headaches, by placing them (with their informed consent) on daily oral administration of levetiracetam. In this small-scale open-label trial, his patients reported a substantial reduction in the frequency and/or severity (intensity, duration, etc.) of their migraine headaches.

Subsequently, the Applicant decided to prepare levetiracetam in an injectable liquid form, and test it for treating migraine headaches that had already begun to emerge, or that had become full-blown. The results were highly positive, as summarized below.

Accordingly, the Applicant discloses herein that orally-ingested levetiracetam is effective in reducing the frequency, intensity, duration, and other adverse effects of migraine headaches, in at least some patients, including patients who do not receive adequate relief from any other known drugs, and patients who cannot take various other drugs because of other medical problems or conditions. Recommended dosages that can be evaluated for any particular patient typically will range from about 250 to about 1500 mg/day, and most patients who respond are likely to show substantial benefits in a dosage range of about 500 to 1000 mg/day.

The Applicant also discloses herein that injectable levetiracetam is highly effective and beneficial in treating and in most cases completely resolving and eliminating acute migraine headaches, in at least some patients, even among patients who could not be treated adequately by triptans or any other known drugs.

As noted below, one patient was treated for a combination of cluster headaches and migraines, with resolution of both. Accordingly, in view of these results, it is anticipated that levetiracetam (in either chronic oral form, for prophylactic purposes, or injected form for acute treatments) will be able to provide substantial headache relief for at least some patients who suffer from cluster headaches, recurring tension-type headaches, and possibly-other types of recurrent acute headaches, as can be determined for any particular patient or class of patients by routine testing.

Since levetiracetam appears to be more benign than a number of other injectable agents that are in widespread use today, and since it has fewer reported side effects (and fewer potential dangers, among certain classes of high-risk patients who suffer from recurrent migraines), it appears to offer (in both oral form, and injectable form) a potentially important new form of treatment that may be highly useful in clinical practice, for treating recurrent migraines, and possibly for treating various other types of recurrent headaches as well, including cluster headaches, recurrent tension headaches, etc.

In addition to disclosing the treatment methods described, this invention also discloses:

(i) a composition of matter, comprising a sterile aqueous injectable formulation that contains levetiracetam as an active therapeutic agent;

(ii) an article of manufacture, comprising a sealed vial which contains a sterile aqueous injectable formulation that contains levetiracetam as an active therapeutic agent; and,

(iii) an article of manufacture of comprising a sealed vial that contains a sterile aqueous injectable formulation that contains levetiracetam, packaged with printed instructions that indicate the levetiracetam is useful for treating at least one type of recurrent acute headache.

### EXAMPLE 1

### Testing of Oral Levetiracetam as a Prophylactic Agent

A limited number of patients who were being treated by the Applicant for recurrent migraine headaches were placed on a regimen of daily oral levetiracetam, in a small-scale open-label trial. All patients were initially tested on a regimen of 250 mg tablets, to ensure that they did not suffer from any adverse reactions; if they did not respond well (or only responded partially) to that dosage, they were given the option of increasing their dosage to 500 or 750 mg tablets. In general, the recurrent migraine patients who were chosen for this type of experimental trial were those who had not responded adequately to various other known candidate treatments. Patients kept daily records of the frequency of their migraine headaches, and also kept logs that recorded the severity of each headache, on a subjective scale of 1 to 10, with 1 being mild, and 10 being extremely intense pain.

A substantial fraction of these patients reported substantial benefits, in reduced frequency and/or reduced severity of their migraine headaches, after commencing chronic oral treatment using levetiracetam.

### EXAMPLE 2

### Preparation of Injectable Levetiracetam

Levetiracetam tablets, purchased commercially, were crushed using a Wedgwood mortar and pestle, forming a total weight of 20 to 40 grams of active ingredient, calculated from the number of tablets. 85 ml of sterile water was added. The entire mixture was allowed to stand for 8 hours and then filtered through a 1 micron filter. The filtrate was then heated to 70° C., and filtered five more times through a 1 micron filter; then, successive filtrations, through a 45 millimicron filter were accomplished, for 5 more times. The resulting solution was buffered to pH 6.0 with either 10% NaOH or 1 normal HCl. Sufficient sterile water was added to make a final volume of 100 ml.

The rationale behind this extraction process is that most of the inactive ingredients in the binder material that was used to make the tablets were eliminated in the cold extraction; hydroxypropylmethylcellulose is insoluble in hot water, and therefore was eliminated with heating.

In a clean room with a sterile laminar flow horizontal hood, the above solution was further filtered through a 22 millimicron filter into a sterile serum vial. The final solution was clear and transparent with a very low viscosity, with FDC blue and yellow colors washed out with the repeated filtrations as above.

Initially, a concentration of 200 mg/ml levetiracetam was compounded in 10 ml multi-dose vials; after several patients were successfully treated, a concentration of 400 mg/ml was adopted for routine use in subsequent tests. The final levetiracetam solution was preservative-free, and, therefore was kept in a refrigerator at 3-4 degrees F. A 90-day expiration date was assigned to all vials of levetiracetam, whether used or not.

### EXAMPLE 3

### Testing of Injectable Levetiracetam For Acute Migraines

Sterile, preservative-free liquid preparations of levetiracetam, at concentrations of 200 mg/ml or 400 mg/ml, was prepared in a laminar flow hood by a compounding pharmacist. Particulate matter was filtered out through a series of filtrations and the solution was stabilized at a pH of 6.0. The resulting solution was clear.

29 patients were treated (19 female and 10 male), and a total of 42 intractable migraines were treated in this study. Their age range was 26 to 59 (mean age, 44 years). All patients had failed treatment with their usual migraine-specific or abortive therapies. Most patients (n=24) were in the second or third day of intractable migraine headache. 5 patients were treated on more than one occasion (1 patient was treated 6 separate times; 1 patient, 5 times, 1 patient, 3 times; and 2 patients, twice each). 6 patients (21%) were already on levetiracetam orally at the time of intravenous treatment. 23 were naive to levetiracetam at the time of treatment.

One patient was treated for a combination of cluster headaches and migraines, with resolution of both.

Patients were treated in the setting of a headache clinic. An antecubital line was started and pulse oximetry monitoring was employed throughout treatment. No other medications were given orally or intravenously to treat the migraine.

In a typical treatment for a first-time patient, a test dose of 400 mg levetiracetam was given, and then 400-600 mg was given every 5 minutes, until maximum effect was reported by the patient. The average dosage given intravenously was 5940 mg (range 400 to 16,000 mg). The average time to best response after treatment was 66 min (range=20-120 min).

Side effects were minimal. 3 patients reported transient nausea, and 2 reported drowsiness, but these side effects dissipated spontaneously after about 30-90 minutes, and they may have been side effects of their migraine status rather than the drug treatment.

Prior to treatment, migraine severity (reported by each patient on a 0 to 10 numeric rating scale (NRS), with 10 representing the absolute worst possible pain) was 8.4. After treatment, it was 1.6, which represents a mean reduction of 81% in migraine severity. 16 patients (55%), representing 21 of 42 episodes, had complete resolution of their ongoing migraine, to a reported severity of zero. Only one patient had no reported response to the treatment.

Accordingly, this preliminary open-label study, using intravenous levetiracetam in the treatment of acute migraines, showed very good efficacy, and was comparable to other acute treatment agents that are widely used.

### REFERENCES

Andersson, K. E., et al, “Beta-adrenoceptor blockers and calcium antagonists in the prophylaxis and treatment of migraine,” Drugs 39(3): 355-73 (1990)

Greenberg, D. A., “Calcium channel antagonists and the treatment of migraine,” Clin Neuropharmacol 9(4): 311-28 (1986)

Kim, K. E., “Comparative clinical pharmacology of calcium channel blockers,” Am Fam Physician. 43(2): 583-8 (1991)

Montastruc, J. L., et al, “Calcium channel blockers and prevention of migraine,” Pathol Biol (Paris) 40(4): 381-8 (1992)

1. A method of reducing recurrent acute headaches in a patient in need of such treatment, comprising oral administration of levetiracetam in a daily dosage that is effective in reducing at least one adverse trait of recurrent acute headaches in said patient.

2. The method of claim 1, wherein levetiracetam is administered orally in a daily dosage of about 250 to about 1500 mg/day.

3. The method of claim 1, wherein levetiracetam is administered orally in a daily dosage of about 500 to about 1000 mg/day.

4. A method of treating an acute headache in a patient in need of such treatment, comprising intravenous injection of levetiracetam in a dosage that is effective in reducing at least one adverse trait of such acute headache in said patient.

5. The method of claim 4, wherein levetiracetam is injected intravenously.

6. The method of claim 4, wherein levetiracetam is injected in an aqueous formulation.

7. The method of claim 4, wherein the patient suffers from recurrent acute headaches.

8. A composition of matter, comprising a sterile aqueous injectable formulation that contains levetiracetam as an active therapeutic agent.

9. An article of manufacture, comprising a sealed vial which contains a sterile aqueous injectable formulation that contains levetiracetam as an active therapeutic agent.

10. The article of manufacture of claim 9, wherein the sealed vial is packaged with printed instructions that indicate the levetiracetam is useful for treating at least one type of recurrent acute headache.
